(19) United States
(12) Patent Application Publication
(10) Pub. No.: US 2008/0147148 A1
(43) Pub. Date: Jun. 19, 2008

(54) Device For Human Body Treatment By Electromagnetic Waves

(76) Inventor: Marcello Rinaldo Baldacchini, Brescia (IT)

Correspondence Address: KILYK & BOWERSOX, P.L.L.C., 400 HOLIDAY COURT, SUITE 102, WARRENTON, VA, 20186, US

(21) Appl. No.: 11/957,686

(22) Filed: Dec. 17, 2007

Related U.S. Application Data
(63) Continuation of application No. PCT/IT2005/000350, filed on Jun. 17, 2005.

(51) Int. Cl. A61B 18/18

(52) U.S. Cl. 607/90

(57) ABSTRACT

The object of the present invention is a device (1) for human body treatment, for example the treatment of alopecia, rheumatic disorders, or for stimulating the vasodilatation of the penis capillaries. The device comprises a seating body (2), a handling case (20), a plurality of separate emitters (30) suitable for emitting a wave having a wavelength equal to the silicon characteristic wavelength, that is, equal to 905 nanometres. An assembly for human body treatment comprises the device and a plurality of bags usable for preventing contact between the emitters and the part of the body to be treated.

### TECHNICAL FIELD OF THE INVENTION

The present invention relates to a device for human body treatment by electromagnetic waves and to a method for carrying out said treatment.

### BACKGROUND OF THE INVENTION

Several solutions are known relating to laboratory devices for human body treatment by waves in the nIR (near Infra-red) or IR (Infra-red) range, generated by laser. Such devices are generally very bulky, expensive, requiring skilled personnel, and therefore are not suitable for home use, for example every day.

There are also solutions relating to devices suitable for home use, generally comprising a brush to move on the part of the body to be treated.

Solutions of the type described above are for example described in documents WO97/09847, RU2231353, DE4012854, WO2004/075985, WO2005/016454, CN1369239, CB2368020, U.S. Pat. No. 5,336,159, EP1005880, DE4143168, U.S. Pat. No. 4,680,822, RU2232610, DE4113803, GB2212010, WO02/053224 and TW580916.

However, known solutions exhibit several disadvantages.

### SUMMARY OF THE INVENTION

The object of the present invention is to overcome the disadvantages of prior art devices and to provide a device for the therapeutic treatment of human or animal body, particularly suitable for a home use.

### BRIEF DESCRIPTION OF THE DRAWINGS

FIG. 1 shows a perspective view of the device according to the present invention.

FIG. 2 shows a plan view of a seating body of device of FIG. 1.

FIGS. 3a and 3b show plan views of a handling case of device of FIG. 1.

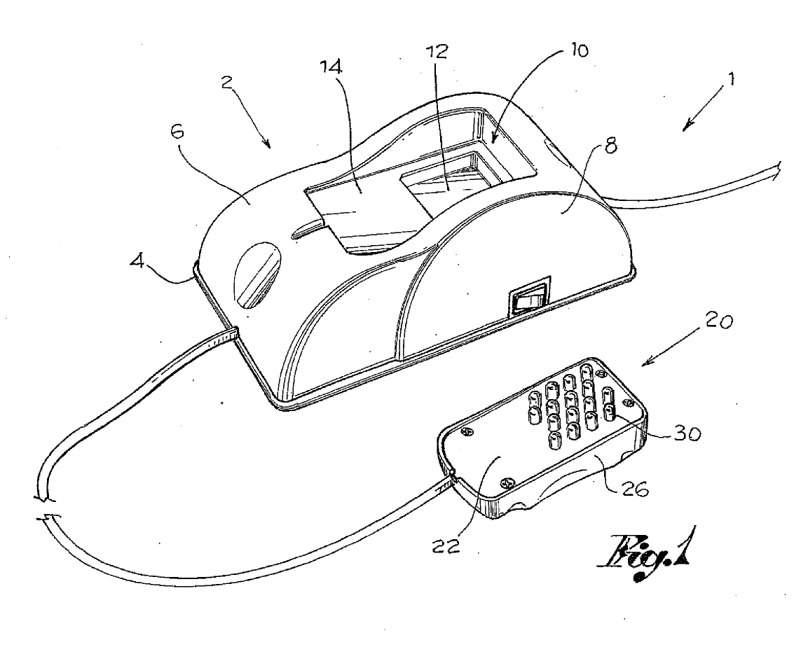

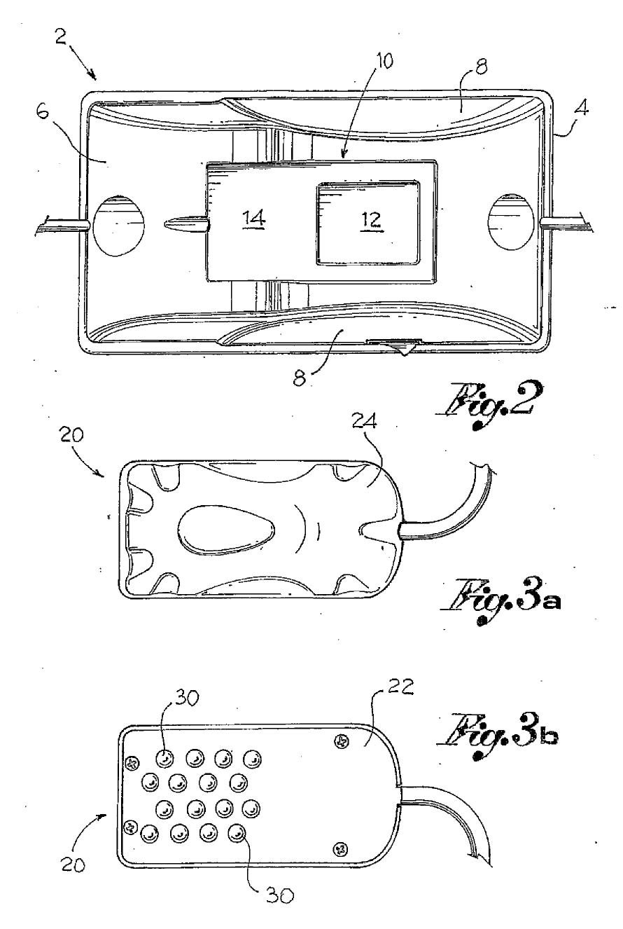

### DESCRIPTION OF THE PREFERRED EMBODIMENTS

With reference to the annexed figures, reference numeral 1 globally indicates a device for human body treatment by electromagnetic waves, said device being suitable for being used on a part of said body to be treated.

Device 1 comprises a seating body 2 suitable for resting on a holder.

According to a preferred embodiment, said body 2 is a box structure internally provided with a space. Said box structure comprises a support base 4, suitable for resting on said holder, an upper wall 6, opposed to said support base, and side walls 8. Said support base 4, said upper wall 6 and said side walls 8 delimit the inside space.

According to a preferred embodiment, said upper wall 6 of body 2 exhibits a seat 10, wherein a lower space 12 and an upper space 14 can be distinguished.

According to a preferred embodiment, said device 1 comprises power supply means. For example, said power supply means are suitable for being connected to the mains for powering said device. In accordance with an embodiment variation, said power supply means comprise at least one battery, for example of the “rechargeable” type, for powering device 1.

Moreover, device 1 comprises a handling case 20, operatively connected to said seating body 2 and suitable for being held in a hand for being handled and for carrying out said treatment.

According to a preferred embodiment, said handling case 20 exhibits a front surface 22, intended for facing the part to be treated during a treatment, a back surface 24, opposed to said front surface, and side surfaces 26.

Moreover, said device 1 comprises substantially monochromatic emission means suitable for emitting a substantially monochromatic wave.

In particular, said emission means comprise a plurality of separate emitters 30, each emitter being suitable for emitting a wave having a predetermined wavelength.

Preferably, said emitters are suitable for emitting an electromagnetic wave having a wavelength equal to the characteristic wavelength of silicon emission, that is, substantially equal to 905 nanometres.

Within the scope of this invention, the term “substantially equal” indicates that, for intrinsic technological constraints, the value of the wavelength emitted by an emitter may differ from the indicated value, but will in any case remain around said value. In particular, according to an embodiment, said wavelength is comprised in a range between 875 nanometres and 935 nanometres.

Preferably, said emitters are light emitting diodes (LED).

Emitters 30 are arranged on said case 20, for impinging at least partly, with said waves, the part of the body to be treated, during the treatment.

Preferably, said emitters 30 are projecting from said front surface 22 of case 20 and spaced from each other.

In particular, emitters 30 are arranged so as to generate a matrix wherein rows and columns can be identified. Preferably, emitters 30 of a row are arranged in an intermediate position relative to the emitters of an adjacent row, with adjacent row or adjacent column to a previous row/column meaning the row/column directly next to the previous row/column.

In particular, an emitter 30 not arranged on a peripheral row or on a peripheral column of the matrix, with peripheral row/column meaning the lat row/column of the matrix, that is, a row/column that, at least on one side, has no adjacent row/column, exhibits a constant distance relative to adjacent emitters.

In other words, said emitters 30 exhibit a honeycombed arrangement.

Said seating body 2 is suitable for seating, at least partly, said case 20, for keeping said device in a non-use condition. In particular, said lower space is suitable for seating said emitters 30, whereas said upper space is suitable for seating said case.

According to a preferred embodiment, said device 1 comprises primary supply means and secondary supply means.

Said secondary supply means seated into said case 20, are suitable for powering said emission means and in particular, said emitters. Preferably, said secondary supply means are suitable for powering said emitters with a predetermined power supply frequency, preferably matching the mains frequency.

On the other hand, said primary supply means, operatively connectable to said power supply means, are seated into said body 2 and operatively connected to said secondary supply means.

In the standard use of the device described above, the device is connected to the mains or powered by battery.

When the device is switched on, said emitters emit a wave falling in the nIR range, preferably equal to the characteristic silicon wavelength, that is, substantially equal to 905 nanometres.

The emission of a wave at the above wavelength can be testable using a special detecting device. nIR waves, in fact, are not within the visible spectrum and therefore are not perceived by the human eye. However, as detecting device it is possible to use a digital camera of the type available on the market, whose lens is sensitive to nIR waves.

Staring at the emitters with naked eye, when the device is operating, it will therefore not be possible to detect any light. On the other hand, framing the emitters with the camera, a light corresponding to the emission of the above wave will be visible.

For the human body treatment, the case is moved close to the part of the body to be treated, aiming the emitters at the part of the body to be treated.

Moving the case on the part to be treated, in contact with, lightly touching or a little away from, the skin, the emitted waves impinge the part to be treated, causing a resonant effect in the cells forming the part to be treated.

The electromagnetic waves generated, having a wavelength substantially equal to the silicon wavelength, can be used for treating alopecia, rheumatic disorders, for dermatological and aesthetic treatments and the like. Such use, moreover, has shown considerable results for obtaining the vasodilatation of the penis capillaries.

Advantageously, the arrangement of the emitters on the front surface of the case allows obtaining an even distribution of the intensity of the waves impinging the part of the body to be treated. Advantageously, such feature allows not causing any undesired reactions on the part of the body to be treated, for example nerve stressing due to an excessive difference of electrical potential generated between adjacent portions of the part of the body to be treated.

Advantageously, moreover, when not temporarily used, for example during a treatment, the device is suitable for being arranged in a non-use configuration wherein the emitters are seated in the lower space of the seat obtained in the seating body. In this way, the emitted waves are confined into said seat.

Advantageously, moreover, it has been found that the use of the described device on a part of the body where a cream or oil or in general, a substance reacting with said part of the body has been applied before and/or after, for the therapeutic or aesthetic treatment thereof, considerably stimulates the reaction of said substance with said part of the body.

The device according to the present invention is preferably marketed as a part of an assembly comprising said device and at least one protective cover.

According to a preferred embodiment, said protective cover comprises a deformable bag suitable for seating, at least partly, said case 20. Preferably, said bag is transparent to the emitted waves and is made of a plastic material, for example polyethylene.

Before carrying out the treatment, case 20, or the part of said case exhibiting emitters 30, is inserted into said bag and then moved close to the part of the body to be treated.

At the end of the treatment, the case is removed from the bag, which is disposed.

Advantageously, the assembly allows using the device for carrying out different treatments on different parts of the body or on different users.

In particular, the assembly allows using specific creams or products for each part of the body, avoiding soiling the emitters and consequently, polluting other parts of the body treated afterwards. Of course, this also allows use by different users, protecting hygiene.

In other words, a portion of the bag provides a wall transparent to the waves, for preventing physical contact between said emitters and said part of the body to be treated.

Advantageously, moreover, said bag also provides a further electrical and chemical insulation between the device and the part of the body to be treated.

Finally, according to a further advantageous aspect thereof, said power supply of said emitters at the main frequency enhances the wave emission, decreasing the emission time, allowing a penetration of said waves into the part of the body to be treated.

1. A device for human or animal body treatment, said device being suitable for being used on a part of said body to be treated and comprising:
a seating body suitable for resting on a holder;
a handling case, operatively connected to said seating body and suitable for being held in a hand for being handled and for carrying out said treatment;
emission means, said emission means comprising a plurality of separate emitters (30), each emitter being suitable for emitting a wave having a wavelength comprised in the range 875-935 nanometres,
wherein said emitters are arranged on said case, for impinging with said waves, during said treatment, said part of the body to be treated,
wherein the emission means are substantially monochromatic,
wherein said emitters are arranged so as to generate a matrix wherein rows and columns can be identified,
and wherein the emitters of a row are arranged in intermediate position relative to the emitters of an adjacent row.

2. A device according to claim 1, wherein said emitters are suitable for emitting a wave having a wavelength substantially equal to the silicon emission wavelength.

3. A device according to claim 2, wherein said emitters are suitable for emitting a wave having a wavelength substantially equal to 905 nanometres.

4. A device according to claim 1, wherein said handling case exhibits a front surface, intended for facing the part to be treated during a treatment, and a back surface, said emitters being arranged on the side of said front surface.

5. A device according to claim 4, wherein said emitters are projecting from said front surface.

6. A device according to claim 1, wherein said emitters are spaced from one another.

7. A device according to claim 1, wherein an emitter not arranged on a peripheral row or on a peripheral column, exhibits a constant distance relative to adjacent emitters.

8. A device according to claim 1, wherein said emitters exhibit a honeycombed arrangement.

9. A device according to claim 1, wherein said seating body is suitable for seating, at least partly, said case, for keeping said device in a non-use condition.

10. A device according to claim 9, wherein said seating body exhibits a support base, suitable for resting on said holder, and an upper wall, opposed to said support base, wherein said upper wall exhibits a seat for seating said case.

11. A device according to claim 10, wherein said seat exhibits a lower space and an upper space, that can be distinguished from each other, said lower space being suitable for seating said emitters and said upper space being suitable for seating, at least partly, said case.

12. A device according to claim 1, comprising secondary supply means suitable for powering said emitters for emitting said wave.

13. A device according to claim 12, wherein said power supply means are seated into said case.

14. A device according to claim 1, comprising primary supply means, suitable for cooperating with said secondary supply means, for powering said secondary supply means.

15. A device according to claim 14, wherein said primary supply means are seated into said seating body.

16. A device according to claim 12, wherein said secondary supply means are suitable for powering said emitters powering them at a predetermined frequency.

17. A device according to claim 16, wherein said secondary supply means are suitable for powering said emitters powering them at a frequency equal to the mains frequency.

18. A device according to claim 1, wherein said emitters are light emitting diodes.

19. An assembly for human or animal body treatment, comprising:
a device for human or animal body treatment, comprising:
a) a seating body suitable for resting on a holder;
b) a handling case, operatively connected to said seating body and suitable for being held in a hand for being handled and for carrying out said treatment;
c) emission means, said emission means comprising a plurality of separate emitters (30), each emitter being suitable for emitting a wave having a wavelength comprised in the range 875-935 nanometres,
wherein said emitters are arranged on said case, for impinging with said waves, during said treatment, said part of the body to be treated,
wherein the emission means are substantially monochromatic,
wherein said emitters are arranged so as to generate a matrix wherein rows and columns can be identified,
and wherein the emitters of a row are arranged in intermediate position relative to the emitters of an adjacent row;
at least one wall transparent to waves, for preventing physical contact between said emitters and said part of the body to be treated.

20. An assembly according to claim 19, wherein said transparent wall is a portion of a bag.

21. An assembly according to claim 19, wherein said wall is made of polyethylene.

22. A method for the treatment of a part of the human or animal body, said method being carried out with a device obtained according to claim 1, wherein said method comprises the steps of:
generating a plurality of monochromatic waves having a wavelength equal to the silicon characteristic wavelength;
moving said case close to the part of the body to be treated;
impinging said part of the body with said waves.

23. A method according to claim 22, further comprising the step of inserting a wall transparent to waves between said emitters and said part of the body to be treated, for preventing physical contact between said emitters and said part of the body to be treated.

24. A method according to claim 23, wherein said insertion step comprises a step of inserting at least partly said case into a bag.

25. A method according to claim 22, further comprising the step of applying a substance, such as a cream or oil, to said part of the body.

26. A method according to claim 25, wherein said application step is carried out before said moving close step.

27. A method according to claim 25, wherein said application step is carried out after said moving close step.

28. A method to treat alopecia on a part of a body comprising impinging said part of body with electromagnetic waves having a characteristic wavelength equal to the silicon emission wavelength for the treatment of alopecia.

29. A method to stimulate a penis erection comprising impinging with electromagnetic waves having a characteristic wavelength equal to the silicon emission wavelength for stimulating the penis erection.

30. A method to treat rheumatic disorders on a part of a body comprising impinging said part of body with electromagnetic waves having a characteristic wavelength equal to the silicon emission wavelength for the treatment of rheumatic disorders.

31. A method of dermatologically and/or aesthetically treating skin comprising impinging said skin with electromagnetic waves having a characteristic wavelength equal to the silicon emission wavelength for the dermatological and/or aesthetic treatment of the skin.

32. A method for stimulating a reaction of substances with a part of the body to be treated comprising impinging said part of body with electromagnetic waves having a characteristic wavelength equal to the silicon emission wavelength in combination with the use of substances reacting with said part of the body to be treated for stimulating the reaction of said substances with said part of the body.
